(19) United States
(12) Patent Application Publication
(10) Pub. No.: US 2026/0216505 A1
(43) Pub. Date: Jul. 30, 2026

(54) SYSTEM AND METHOD FOR DELIVERY OF NUCLEIC ACID MOLECULES BY IONTOPHORESIS

(71) Applicant: CONTINUITY BIOSCIENCES, LLC, Cary, NC (US)

(72) Inventors: Maria P. Torres, Rolesville, NC (US); Raquel Maeso-Diaz, Raleigh, NC (US); Leigh Dement, Clayton, NC (US); Brendan Berkel, Raleigh, NC (US); Casey Haigh, Cary, NC (US); William Daunch, Cary, NC (US)

(21) Appl. No.: 19/459,991

(22) Filed: Jan. 26, 2026

Related U.S. Application Data
(60) Provisional application No. 63/749,361, filed on Jan. 24, 2025.

(51) Int. Cl. A61N 1/32; A61K 31/7105; A61N 1/04

(52) U.S. Cl. CPC A61N 1/325; A61K 31/7105; A61N 1/0436; A61N 1/0444

(57) ABSTRACT

A method delivers ribonucleic acids to a target site of internal body tissue using iontophoresis. A ribonucleic acid drug solution having a concentration of from about 10 mg/mL to about 40 mg/mL is disposed in a surgically implantable iontophoresis device including a source electrode and a counter electrode. The device comprises a housing adapted to be secured to the target tissue. A pad in the housing absorbs and retain aqueous formulations of nucleic acids prior to iontophoretic delivery. A buffer gel maintains pH. A localized electrical field occurs by applying a current of between about 1 mA to about 10 mA at the target site for inducing the drug solution of nucleic acid to be delivered by iontophoresis into the tissue.

### CROSS-REFERENCES

This application claims the benefit of U.S. Provisional Patent Application No. 63/749,361, filed Jan. 24, 2025, the contents of which is hereby incorporated by reference herein in its entirety for all purposes.

### BACKGROUND

A medical system and method for interventional drug delivery are described and, more particularly, a system and method are described for using iontophoresis for targeted delivery of nucleic acid molecules and other genomic medicines.

Among novel genomic medicines with promising potential, mRNA therapeutics are at the forefront for targeted treatments. However, delivering these molecules effectively to target cells, without eliciting undesirable immune responses remains a significant challenge. Iontophoresis can deliver genomic medicines directly into affected organs and other parts of the body. The delivery of mRNA molecules by iontophoresis represents a promising alternative to achieve targeted delivery to a specific organ, potentially minimizing off-target effects and extending therapeutic kinetics in comparison to standard delivery methods.

An iontophoresis device that implants directly onto an organ may be used to infuse genomic medicines such as siRNA, mRNA, and the like, into the organ. The device uses iontophoresis to drive the therapeutic genomic medicines into the organ using electrical currents that pass through a drug solution into the tissue. The device includes an implantable reservoir containing the drugs to be delivered and an electrode. The reservoir is implanted on the organ and may be connected through the abdomen to an infusion pump and electrical leads from the electrode. The circuit is completed by a second electrode placed on the body of the patient for generating an electrical field. Electromotive and electro-osmotic forces cause the genomic medicines to pass across the organ surface and into the tissue.

A suitable implantable iontophoresis device including a reservoir is described in Applicant's co-pending U.S. patent applications. One application is entitled Implantable Reservoir for Use with a Medical Device and System for Interventional Drug Delivery, published as U.S. Published Application No. 2022/0218978. Another is U.S. application Ser. No. 19/339,069, titled Reservoir for use with a Medical Device and System for Interventional Drug Delivery and filed Sep. 24, 2025. The contents of both applications are incorporated herein by reference herein in their entirety. Similar devices are described in U.S. Pat. No. 10,695,562, titled Interventional Drug Delivery System and Associated Methods, and U.S. Pat. Nos. 10,532,226 and 11,351,399, titled Combined Local Delivery of Therapeutic Agents Using Interventional Devices and Radiation. The contents of all three patents are also hereby incorporated by reference herein in their entirety.

For the foregoing reasons, there is a need for an iontophoresis system and method including an implantable reservoir for use in the delivery of nucleic acids and other genomic medicines directly into a targeted site.

### SUMMARY

A method of delivering ribonucleic acids to a target site of internal body tissue is provided. The method comprises the steps of providing a ribonucleic acid drug solution having a concentration of from about 10 mg/mL to about 40 mg/mL. A surgically implantable iontophoresis device is also provided, including a source electrode and a counter electrode. The iontophoresis device comprises a housing adapted to be secured to the target tissue and defining a reservoir having an opening covered by a membrane. A pad is disposed in the reservoir to absorb and retain aqueous formulations of nucleic acids prior to iontophoretic delivery. A buffer gel maintains pH in the reservoir. A localized electrical field is generated by applying a current of between about 1 mA to about 10 mA between the source electrode and the counter electrode at the tissue of the target site for inducing the drug solution to permeate the membrane. The nucleic acid is delivered by iontophoresis into the tissue of the target site.

In one aspect, the source electrode comprises platinum. The source electrode may also comprise a carbon based electrode, including carbon cloth, carbon brush, carbon rod, carbon mesh, carbon veil, carbon paper, carbon felt, granular activated carbon, granular graphite, carbonized cardboard, graphite plate, reticulated vitreous carbon, graphite, graphite felt, and platinum black. Alternatively, the source electrode may comprise a metal based electrode, including plate, sheet, mesh and wire selected from aluminum, stainless steel, silver, silver chloride, nickel, copper, gold, titanium, palladium, and platinum.

In one aspect, the buffer comprises TE comprising 10 mM Tris and 1 mM EDTA. In another aspect, the buffer comprises TBE comprising 89 mM TRIS, 89 mM Boric acid and 2 mM EDTA. In yet another aspect, the buffer comprises TAE comprising 40 mM Tris, 20 mM acetic acid and 1 mM EDTA. Further, the buffer may comprise PBS comprising 137 mM sodium chloride, 2.7 mM potassium chloride, 10 mM sodium phosphate dibasic, and 1.8 mM potassium monobasic. The buffer has a pH range of 7-9 and a concentration range of 0.5×-10×.

The step of providing nucleic acids comprises providing mRNA wherein the operating current is 5mA to 8mA and the operating duration is 90 minutes. The step of providing nucleic acids may also comprise providing siRNA wherein the step of generating a localized electrical field comprises applying a current of between about 5 mA to about 10 mA, a current of between about 7.5 mA to about 10 mA, or a current up to about 10 mA.

The target site of the method may be ethidium bromide doped agarose gel tissue surrogate or porcine kidney or porcine liver tissue. mRNA can be delivered at 5 mA for 90 minutes.

### BRIEF DESCRIPTION OF THE DRAWINGS

For a more complete understanding of the method and system for an interventional drug delivery system and method for genomic medicines, reference should now be had to the embodiments shown in the accompanying drawings and described below. In the drawings:

FIG. 1 is photographs showing naked siRNA delivered by iontophoresis into an ethidium bromide doped agarose tissue surrogate.

FIG. 2 is a photograph showing naked siRNA delivery as in FIG. 1 with fine control of siRNA penetration depth and breadth into the agarose tissue surrogate.

FIG. 3 is a graph showing voltage versus time voltage traces for iontophoresis parameters for delivery to porcine liver tissue and porcine kidney tissue.

FIGS. 4A-4C are photographs showing siRNA delivery as in FIG. 1 including back, middle and front cross-sections for three different iontophoresis parameters for delivery.

FIGS. 5A-5C are photographs showing siRNA delivery to a skin pad passively and for two different iontophoresis parameters for delivery.

FIG. 6 is a graph showing voltage versus time voltage traces for iontophoresis parameters for delivery to skin pads as shown in FIGS. 5A-5C.

FIG. 7 is a graph showing voltage versus time voltage traces for iontophoresis parameters for delivery to skin pads under different iontophoresis conditions and electrode materials.

FIG. 8 is a graph showing voltage versus time voltage traces for iontophoresis parameters for delivery to agarose gel capsules and kidney tissue using pads under different iontophoresis conditions.

FIG. 9 is photographs showing a cross-section and top view of non-woven stacks having undergone iontophoresis using three different operating parameters.

FIGS. 10A and 10B are photographs showing iontophoresis delivery of mRNA to an agarose gel model capsule.

FIGS. 11A and 11B is a table and a photograph showing iontophoresis delivery of mRNA to a benchtop agarose gel model.

FIGS. 12A-12E are photographs showing iontophoresis delivery of mRNA and siRNA to an agarose gel model with gold staining.

FIG. 13 shows delivery of mRNA by iontophoresis on a benchtop agarose gel model with ethidium bromide.

FIG. 14 shows fluorescence phase contrast microscopy images of SKRT cells after 24 hr of iontophoresis treatment (top) and diffusion control (bottom).

FIG. 15 shows EGFP-mRNA expression in a porcine kidney cortex treated by iontophoresis versus Diffusion control (*p-value<0.05).

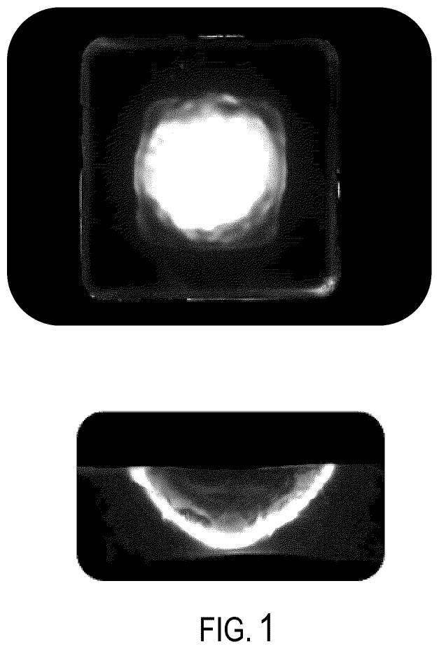

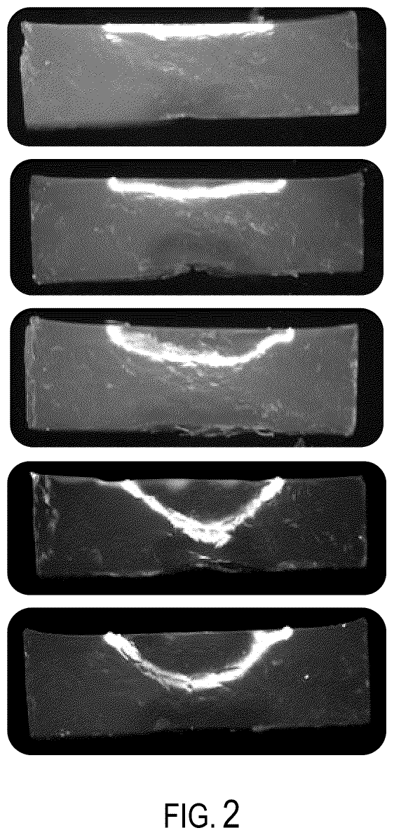

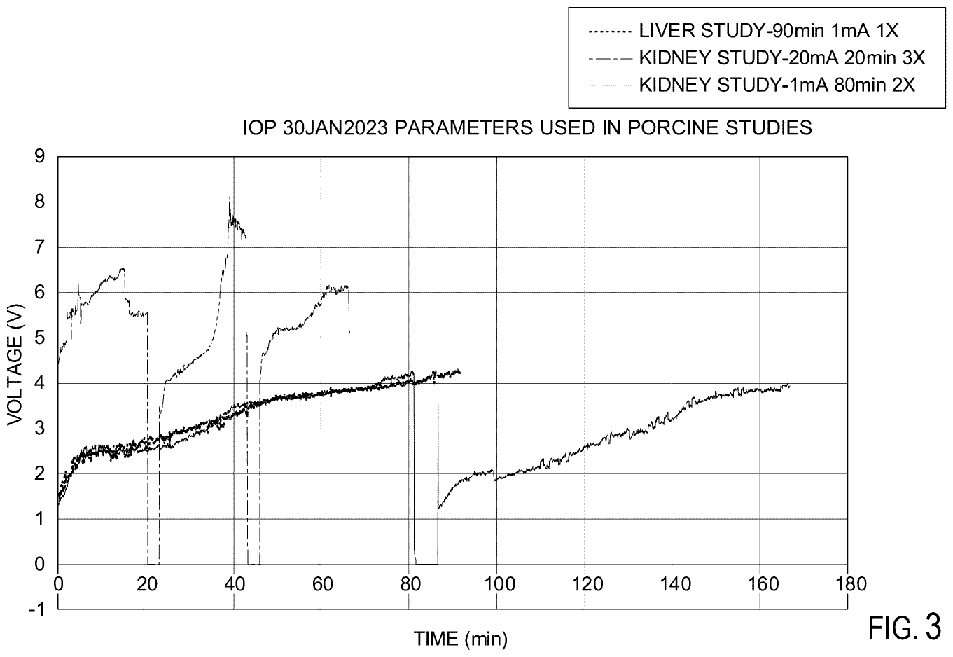

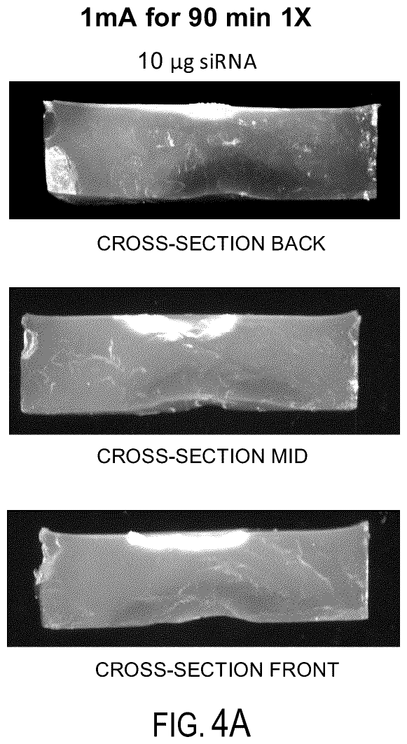

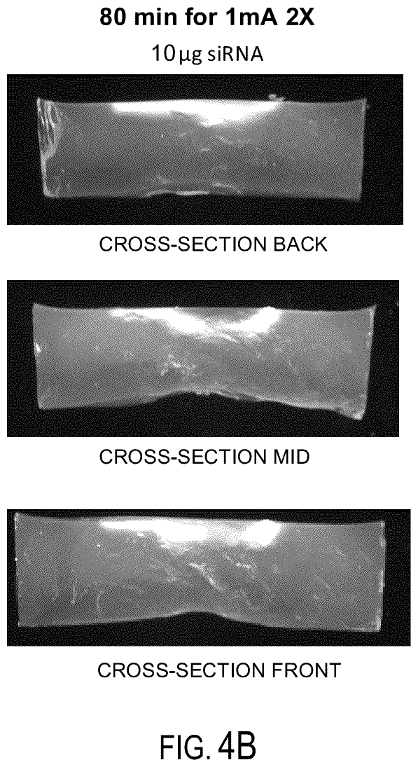

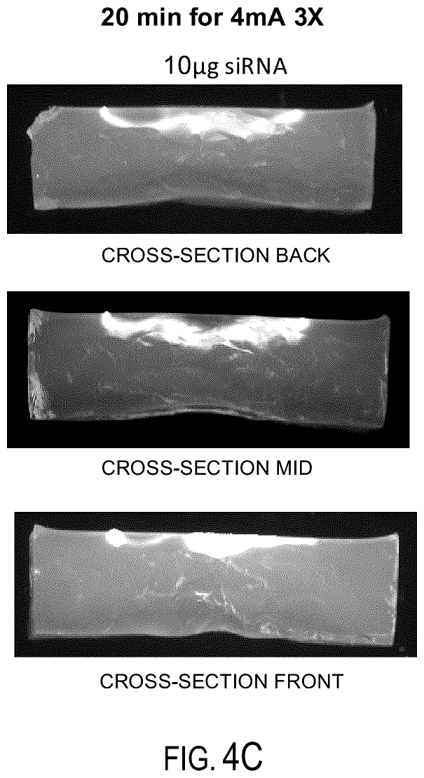

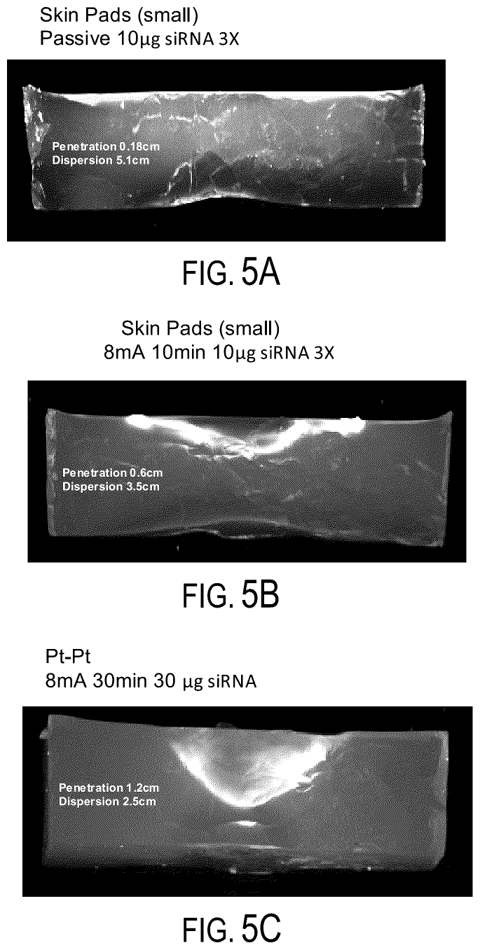

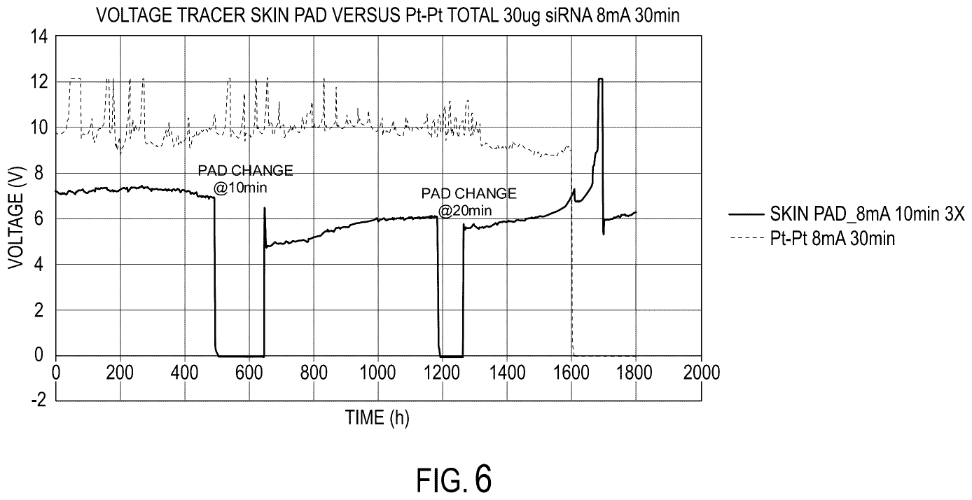

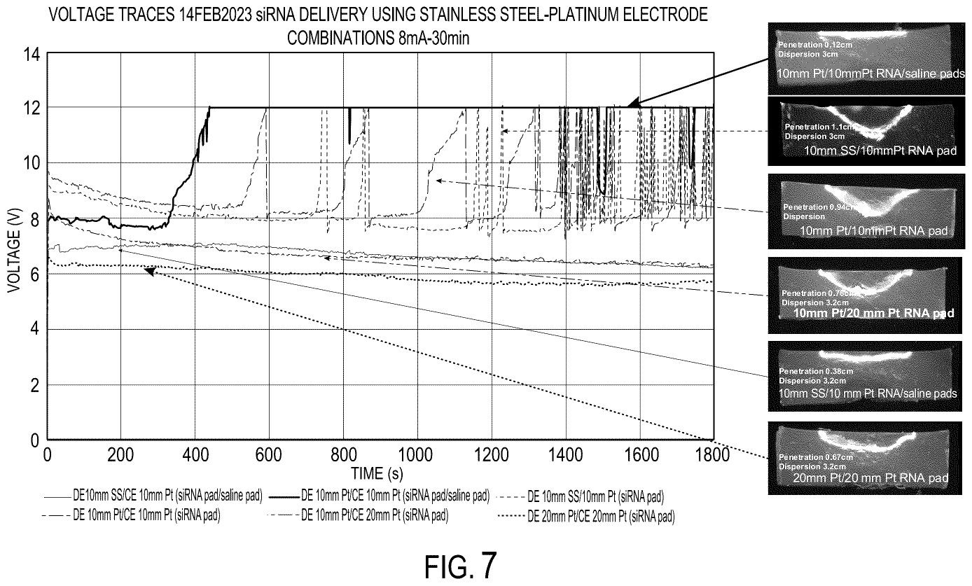

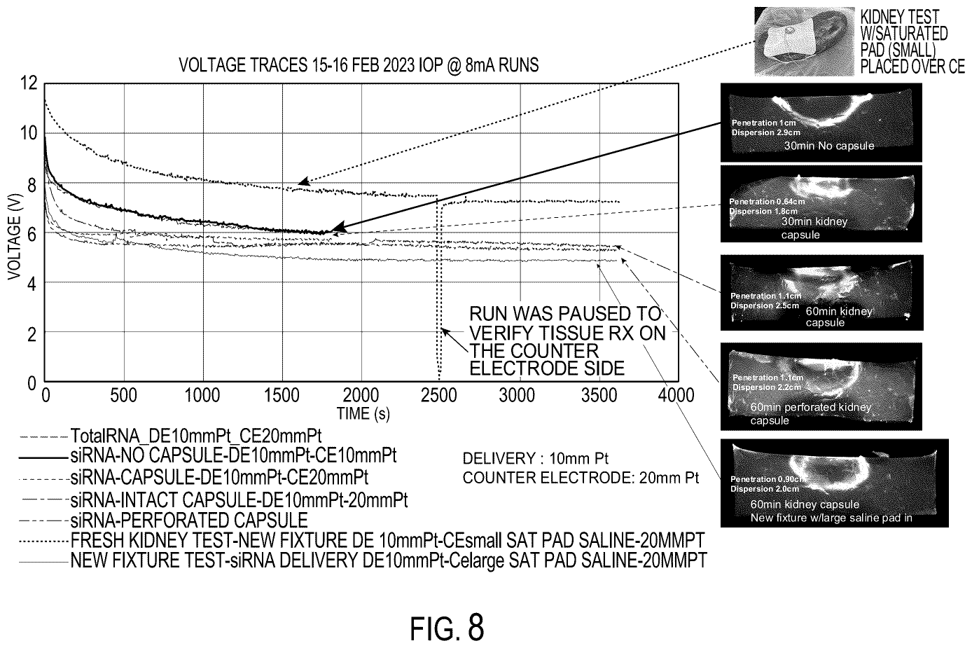

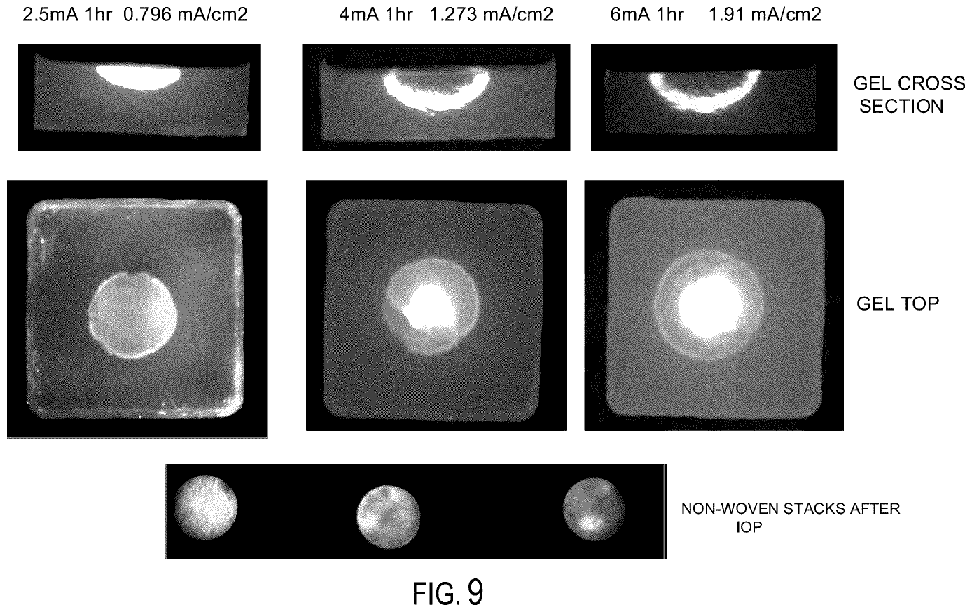

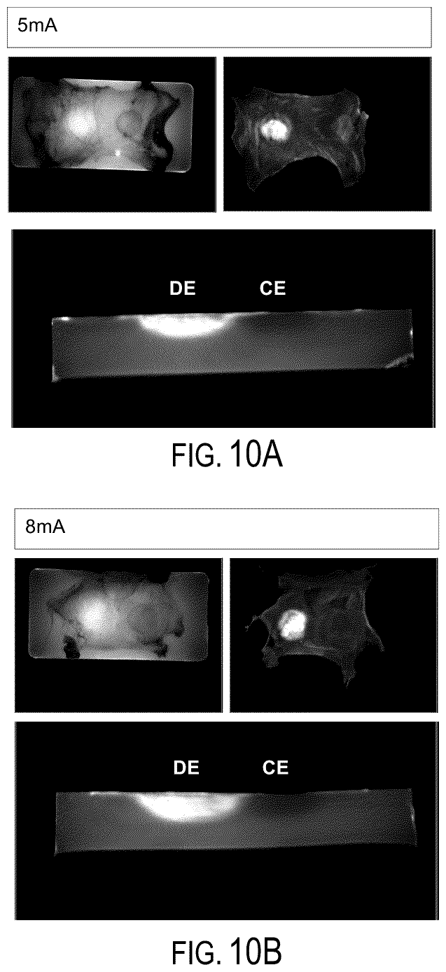

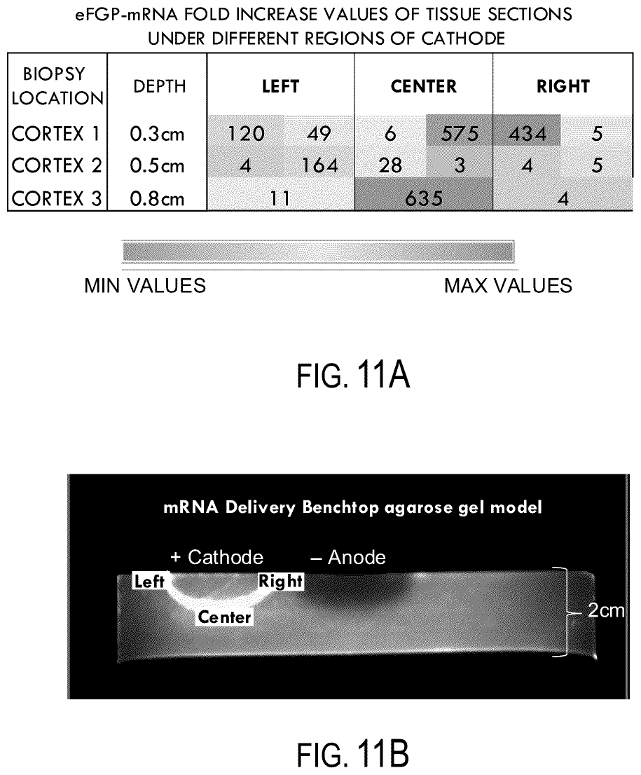

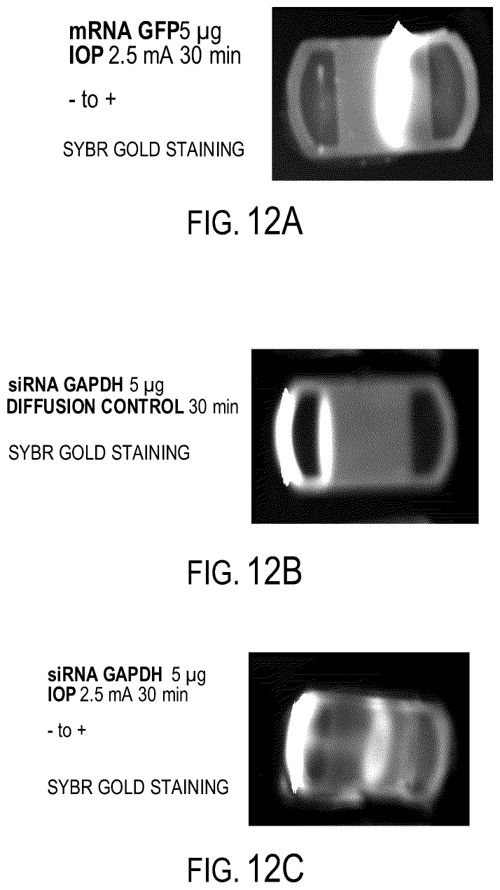

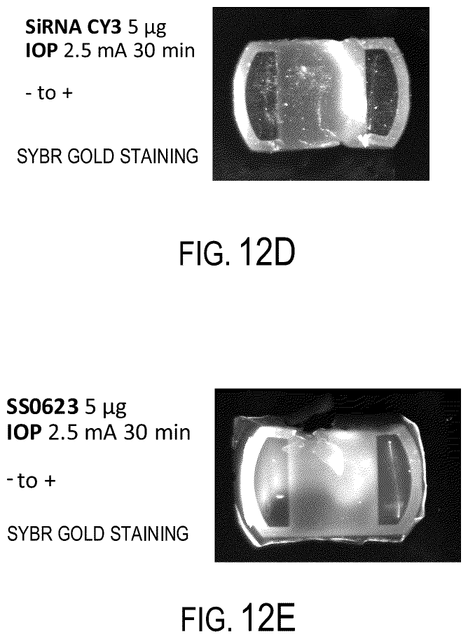

### DESCRIPTION

An implantable medical device that implants directly onto an organ or other tissue may use iontophoresis to infuse genomic medicines directly into the organ, including negatively charged macromolecules such as RNA, siRNA, mRNA, plasmid DNA and oligonucleotide (ODN), and the like. The nucleic acid molecules are highly negatively charged and have high charge-to-molecular weight ratios making them good candidates for iontophoretic delivery. Among the three mechanisms proposed for iontophoresis, electromigration is expected to be the most dominant driving force for these macromolecules.

Iontophoresis drives the therapeutic genomic drugs into the organ using electrical currents that pass through the drug solution into the organ tissue. In one embodiment, the device includes an implantable reservoir containing the drugs and an electrode. The reservoir is implanted on the organ and connected external to the body to electrical leads from the electrode. The circuit is completed by a second electrode placed on the body of the patient for generating an electrical field.

Iontophoresis uses electromotive and electro-osmotic forces which cause drugs from an external source to pass through a membrane and into target tissue. Many factors affect the results of iontophoresis, including physicochemical properties of the compound (molecular size, charge, concentration), drug formulation (types of vehicle, buffer, pH, viscosity, presence of other ions), equipment used (available current range, constant vs. pulsed current, type of electrode), biological variations (tissue site, regional blood flow, age, sex), tissue temperature and duration of iontophoresis. The mechanisms of iontophoretic transport across the membrane include direct interactions of the electric field with the charge of an ionic compound (electromigration), convective solvent flow affecting the transport of both neutral and ionic compounds (electro-osmosis), and electric field-induced pore formation in the membrane referred to as electropermeabilization (EP). Currently, iontophoresis is believed to be carried out through paracellular transport. Enhanced tissue penetration of neutral molecules by anodal iontophoresis and improved penetration of cations through this supplementary driving force are achieved. The iontophoretic efficiency is affected by several factors, including ion and molecule permeability, environmental pH, current density (constant or alternating), shape of the electrodes, duration of treatment, drug properties, and concentration.

An embodiment of an implantable device and system for iontophoretic delivery of therapeutic nucleic acids and other genomic medicines is described in the aforementioned U.S. application Ser. No. 19/339,069. The delivery system is designed to stabilize nucleic acid molecules during iontophoretic transport and to provide consistent, reproducible delivery into a target tissue site in the body. The nucleic acid delivery system comprises a reservoir that houses a nucleic acid pad, a buffer gel, and a platinum electrode. The reservoir functions as the container for the other listed functional components and provides alignment and spacing of the components for proper ionic conduction. In particular, the configuration of the reservoir provides secure positioning of the electrode for application of current. The pad is configured to load, absorb, and retain aqueous formulations of the nucleic acids prior to iontophoretic delivery.

In use, the nucleic acid delivery device is secured to a site of target tissue. The pad is loaded with an aqueous nucleic acid solution and positioned within the reservoir adjacent to a buffer gel. The platinum electrode applies electrical current across the reservoir, driving iontophoretic transport of nucleic acid molecules across the membrane and into the target tissue. The buffer gel stabilizes and maintains local pH during iontophoretic delivery. The pad provides a sustained source of nucleic acids while the semipermeable membrane delivers the nucleic acid to the target tissue. The cooperative interaction of these components enables efficient, controlled, and reproducible delivery of nucleic acid molecules into the target tissue, while preserving molecular integrity of the molecules and ensuring consistent therapeutic outcomes.

The general parameters for nucleic acid delivery to a substrate by iontophoresis include electrical current, duration, electrode material, and buffer system. The substrates investigated included agarose, hydrogel, biocompatible sponge or foam, and body tissue. Operating current varied from 0 mA to 40 mA (0.5-25, 1-20, 2.5-12) over a period of 1 minute to six hours. Operating current was also applied at different levels over the course of treatment, in cycles or pulsed.

For the electrode material, carbon based electrodes can be used, including carbon cloth, carbon brush, carbon rod, carbon mesh, carbon veil, carbon paper, carbon felt, granular activated carbon, granular graphite, carbonized cardboard, graphite plate, reticulated vitreous carbon, graphite, graphite felt, and platinum black. Metal based electrodes are also suitable, including plate, sheet, mesh and wire of aluminum, stainless steel, silver, silver chloride, nickel, copper, gold, titanium, palladium, and platinum. The electrode materials can be porous structures to allow for higher surface areas as well as adsorption of contaminants. Three-dimensional electrodes can also be used to increase the power density and volumetric density of both the anode and cathode-based performances. The electrodes can be coated in materials like titanium nitride, nafion and graphene.

A buffer system for iontophoretic delivery of nucleic acids has been developed to neutralize acid-base chemistry in the cathode/anode. TE is 10 mM Tris and 1 mM EDTA. TBE is 89 mM TRIS, 89 mM Boric acid and 2 mM EDTA. TAE is 40 mM Tris, 20 mM acetic acid and 1 mM EDTA. PBS is 137 mM sodium chloride. 2.7 mM potassium chloride, 10 mM sodium phosphate dibasic, and 1.8 mM potassium monobasic. The pH range for the buffers is 7-9. The concentration range of the buffering system is 0.5×-10×.

Encapsulation agents might be useful for genomic medicine delivery to enhance molecule stability and ideal characteristics (i.e., charged) for electrorepulsive, electroattractive and electroosmotic iontophoretic delivery. These can be cationic, anionic, neutral, lipid-base, polymer-based, single layer and multi-layer systems

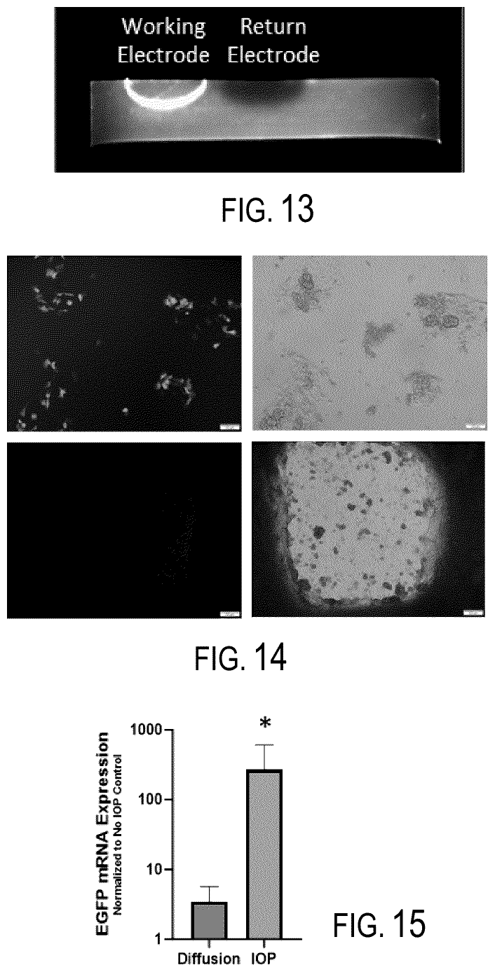

The feasibility of iontophoresis delivery of mRNA was investigated in a benchtop agarose model, in-vitro on porcine kidney cell lines, and in-vivo locally to porcine kidney tissue. The successful delivery of mRNA by iontophoresis was initially demonstrated using the benchtop agarose gel model. Numerous parameters were optimized, including electrode type and material, electrode design and configuration, operating current, and treatment duration. After multiple iterations evaluating these parameters, the optimum delivery of mRNA mimicking a kidney cortex delivery penetration depth was obtained at 5 mA treatment for 90 min (FIG. 13).

The transfection efficiency of iontophoresis delivery of liposome-encapsulated EGFP-mRNA was optimized on PK15 and SKRST porcine kidney cell lines embedded in 3D bioprinted GelMA Alginate hydrogel models. Representative microscopy data clearly show that cells that were treated by iontophoresis expressed green fluorescence (i.e., EGFP) as opposed to cells treated by diffusion alone after 24 hours of treatment (FIG. 14), which was further confirmed by One-step-RT-PCR (data not shown).

Collectively, results from the tissue surrogate models experiments were used to systematically design in vivo studies to deliver mRNA by iontophoresis directly to kidney tissue. An iontophoretic device was placed directly on the kidney of anesthetized Yorkshire female pigs, and iontophoresis treatment was administered at 5 mA for 90 minutes. After a 24-hour recovery and following IACUC guidelines, a necropsy was executed, and fresh kidney tissue biopsy samples were collected and preserved for subsequent analysis. Overall animal health was excellent during and after 24 hours of iontophoresis treatment and no adverse kidney tissue or systemic toxicity reactions were observed. In agreement with results from tissue surrogate models, EGFP-mRNA expression was significantly upregulated in porcine kidney cortex biopsy samples compared with passive diffusion delivery (FIG. 15), as further confirmed by fluorescence microscopy (data not shown). Cumulatively, these results validated the usefulness of surrogate tissue models used in the study by demonstrating that the device configuration selected, and the treatment conditions identified in the surrogate models, were indeed mimicking in-vivo kidney delivery of mRNA, including tissue depth, coverage and transfection efficiency.

Overall, the efficient delivery of functional mRNA by iontophoresis was demonstrated on benchtop, in vitro, and in vivo porcine kidney models. Potential benefits from this novel delivery platform include precise delivery, lower dose administered, greater efficacy, and minimized toxicity. Altogether, taking into consideration that extra-hepatic delivery of mRNA is desired to treat a vast array of common and rare, monogenic, and polygenic diseases, iontophoresis technology represents a promising alternative to viral and non-viral vector delivery approaches to a wide range of solid organs in addition to the liver.

Sample data benchtop agarose model siRNA delivered by iontophoresis technology into ethidium bromide doped agarose tissue surrogate was also successful. siRNA penetration depth and breadth into the agarose tissue was controlled.

mRNA was successfully delivered at different layers of the kidney cortex by iontophoresis. The mRNA fold increase suggests mRNA is delivered in kidney tissue in a similar pattern obtained in the agarose benchtop model, but with a preferential delivery towards the anode.

Delivery of mRNA, siRNA and other nucleic acids is a promising alternative delivery approach for extra-hepatic genomic medicine delivery. Minimally invasive iontophoresis technology for delivering large molecular genomic medicines, both naked and encapsulated, now available Nucleic acids can be delivered alone or in combination with multiple molecules or therapeutics including other nucleic acids, antibodies, small molecule drugs, enzymes, peptides, proteins, among others.

We claim:

1. A method of delivering ribonucleic acids to a target site of internal body tissue, the method comprising the steps of:
providing a ribonucleic acid drug solution having a concentration of from about 10 mg/mL to about 40 mg/mL;
providing a surgically implantable iontophoresis device including a source electrode and a counter electrode, the iontophoresis device comprising
a housing adapted to be secured to the target tissue and defining a reservoir having an opening covered by a membrane,
a pad to absorb and retain aqueous formulations of nucleic acids prior to iontophoretic delivery, and
a buffer gel,
generating a localized electrical field by applying a current of between about 1 mA to about 10 mA between the source electrode and the counter electrode at the tissue of the target site for inducing the drug solution to permeate the membrane,
wherein the nucleic acid is delivered by iontophoresis into the tissue of the target site at a treatment.

2. The method as recited in claim 1, wherein the source electrode comprises platinum.

3. The method as recited in claim 1, wherein the source electrode comprises a carbon based electrode, including carbon cloth, carbon brush, carbon rod, carbon mesh, carbon veil, carbon paper, carbon felt, granular activated carbon, granular graphite, carbonized cardboard, graphite plate, reticulated vitreous carbon, graphite, graphite felt, and platinum black.

4. The method as recited in claim 1, wherein the source electrode comprises a metal based electrode, including plate, sheet, mesh and wire selected from aluminum, stainless steel, silver, silver chloride, nickel, copper, gold, titanium, palladium, and platinum.

5. The method as recited in claim 1, wherein the buffer comprises TE comprising 10 mM Tris and 1 mM EDTA.

6. The method as recited in claim 1, wherein the buffer comprises TBE comprising 89 mM TRIS, 89 mM Boric acid and 2 mM EDTA.

7. The method as recited in claim 1, wherein the buffer comprises TAE comprising 40 mM Tris, 20 mM acetic acid and 1 mM EDTA.

8. The method as recited in claim 1, wherein the buffer comprises PBS comprising 137 mM sodium chloride, 2.7 mM potassium chloride, 10 mM sodium phosphate dibasic, and 1.8 mM potassium monobasic.

9. The method as recited in claim 1, wherein the buffer comprises has a pH range of 7-9.

10. The method as recited in claim 1, wherein the buffer has a concentration range of 0.5×-10×.

11. The method as recited in claim 1, wherein the step of providing nucleic acids comprises providing mRNA.

12. The method as recited in claim 11, wherein the operating current is 5 mA to 8 mA.

13. The method as recited in claim 12, wherein the operating duration is 90 minutes.

14. The method as recited in claim 1, wherein the step of providing nucleic acids comprises providing siRNA.

15. The method as recited in claim 1, wherein the step of generating a localized electrical field comprises applying a current of between about 5 mA to about 10 mA.

16. The method as recited in claim 1, wherein the step of generating a localized electrical field comprises applying a current of between about 7.5 mA to about 10 mA.

17. The method as recited in claim 9, wherein the step of generating a localized electrical field comprises applying a current up to about 10 mA.

18. The method as recited in claim 1, wherein the target site is ethidium bromide doped agarose gel tissue surrogate.

19. The method as recited in claim 1, wherein the target site is porcine liver tissue.

20. The method as recited in claim 1, wherein the target site is porcine kidney tissue.

21. The method as recited in claim 1, wherein mRNA is delivered at 5 mA for 90 minutes.
